(19) United States
(12) Patent Application Publication
(10) Pub. No.: US 2019/0314548 A1
(43) Pub. Date: Oct. 17, 2019

(54) Extracellular Matrix Implant

(71) Applicant: Yichieh Shiuey, San Jose, CA (US)

(72) Inventor: Yichieh Shiuey, San Jose, CA (US)

(73) Assignee: Keramed, Inc., Fairfield, NJ (US)

(21) Appl. No.: 16/343,253

(22) PCT Filed: Nov. 19, 2017
(86) PCT No.: PCT/US17/62449

§ 371 Date: Apr. 18, 2019

Related U.S. Application Data
(60) Provisional application No. 62/425,227, filed on Nov. 22, 2016.

(51) Int. Cl. A61L 27/22; A61L 27/24; A61L 27/20

(52) U.S. Cl. CPC A61L 27/227; A61L 27/24; A61L 27/20; A61L 2300/80; A61L 2430/40; A61L 2300/30; A61L 2300/204; A61L 2430/16; A61L 2300/216

(57) ABSTRACT

A corneal implant includes one or more extracellular matrix (ECM) components; and one or more crosslinking/polymerization promoting agents, wherein the one or more ECM components undergo crosslinking/polymerization on exposure to an initiator.

### CROSS-REFERENCE TO RELATED APPLICATIONS

This application claims the benefit of priority under 35 U.S.C. § 119(e) to U.S. Provisional Patent Application Ser. No. 62/425,227, filed on Dec. 2, 2016, titled “EXTRACELLULAR MATRIX IMPLANT,” the disclosure of which is hereby incorporated by reference in its entirety for all purposes.

### FIELD OF THE INVENTION

The present invention generally relates to implants, and more particularly, to corneal implants, which include a combination of one or more extracellular matrix (ECM) components and one or more crosslinking/polymerization promoting chemical agents that when exposed to an initiator, undergoes crosslinking/polymerization to strengthen and/or shape the implant.

### BACKGROUND OF THE INVENTION

Keratoconus is a progressive eye disease in which the normally round cornea thins and begins to bulge into a cone-like shape. This cone shape deflects light as it enters the eye on its way to the light-sensitive retina, causing distorted vision. As the cornea becomes more irregular in shape, it causes progressive nearsightedness and irregular astigmatism to develop, creating additional problems with distorted and blurred vision, as well as with glare and light sensitivity.

The weakening of the corneal tissue that leads to keratoconus may be due to an imbalance of enzymes within the cornea. This imbalance makes the cornea more susceptible to oxidative damage from compounds called free radicals, causing it to weaken and bulge forward. Risk factors for oxidative damage and weakening of the cornea include a genetic predisposition, explaining why keratoconus often affects more than one member of the same family Keratoconus is also associated with overexposure to ultraviolet rays from the sun, excessive eye rubbing, a history of poorly fitted contact lenses and chronic eye irritation.

In the mildest form of keratoconus, eyeglasses or soft contact lenses may help. As the disease progresses and the cornea thins and becomes increasingly more irregular in shape, glasses and regular soft contact lens designs may no longer provide adequate vision correction. Treatment for progressive keratoconus range from corneal collagen cross-linking to corneal transplant also called a penetrating keratoplasty (PK or PKP).

Various human and animal diseases and conditions involve the weakening or loss of strength of tissues containing ECM components such as collagen. These include keratoconus, eye bags, wrinkles, sleep apnea, pelvic prolapse in women, hernias among others. Therefore, a need exists for compositions, methods and devices to strengthen different types of tissue that contain ECM components.

### BRIEF DESCRIPTION OF DRAWINGS

FIG. 1 illustrates an embodiment of a delivery device containing a mixture of ECM proteins and one or more crosslinking/polymerization promoting agents in a liquid or gel form;

FIG. 2A illustrates an embodiment of a top view of a corneal implant that is partially crosslinked/polymerized and after exposure to an initiator, becomes partially or fully cross linked/polymerized; and

FIG. 2B illustrates an embodiment of a cross-sectional view of a corneal implant that is partially crosslinked/polymerized and after exposure to an initiator, becomes partially or fully crosslinked/polymerized.

### SUMMARY OF INVENTION

The present invention provides compositions and methods for corneal implants. In an embodiment, the implant includes a combination of ECM components and one or more crosslinking/polymerization promoting chemical agents that when partially or fully crosslinked/polymerized, can strengthen and/or shape the implant in the eye.

Thus, in one embodiment, the disclosure provides an implant, which includes one or more extracellular matrix (ECM) components; and one or more crosslinking/polymerization promoting agents, wherein the one or more ECM components undergo crosslinking/polymerization on exposure to an initiator.

In another embodiment, the disclosure provides a method of preparing a corneal implant, which includes mixing one or more extracellular matrix (ECM) components with one or more crosslinking/polymerization promoting agents; and inserting the mixture into an eye of a patient in need thereof, wherein the ECM proteins undergo crosslinking with the one or more crosslinking/polymerization promoting chemical agents on exposure to an initiator.

### DETAILED DESCRIPTION OF THE INVENTION

The present disclosure is to be considered as an exemplification of the invention, and is not intended to limit the invention to the specific embodiments illustrated by the description below. That is, the invention is not limited in its application to the details of construction and the arrangement of the components set forth in the following description or illustrated in the drawings. The invention is applicable to other embodiments or of being practiced or carried out in various ways. It is to be understood that the phraseology and terminology employed herein is for e purpose of description and should not be regarded as limiting. In other modules, well-known methods, procedures, components and circuits have not been described in detail so as not to obscure the teachings of the present disclosure.

In one or more embodiments of the present invention, the disclosure provides an implant and a method of preparing and inserting a corneal implant into an eye of a patient in need thereof. The implant includes a mixture of one or more ECM components; and one or more crosslinking/polymerization promoting chemical agents. When the mixture is exposed to an initiator (e.g. energy) or over a sufficient amount of time, the components of the mixture undergo partial or total crosslinking/polymerization to strengthen and/or shape the implant.

Thus, in one embodiment, the one or more extracellular matrix (ECM) components can undergo crosslinking/polymerization with the one or more crosslinking/polymerization promoting agents on exposure to an initiator. In another embodiment, the one or more ECM components is an ECM protein or a ECM carbohydrate, or a combination thereof. In another embodiment, the one or more ECM components is collagen, heparan sulfate, chondroitin sulfate, keratan sulfate, hyaluronic acid, elastin, fibronectin, laminin, or a combination thereof. In another embodiment, the one or more crosslinking/polymerization promoting agents is a photoreactive agent, a carbohydrate, or a plant extract. In another embodiment, the photoreactive agent is riboflavin or rose bengal and the initiator is ultraviolet light. In another embodiment, the one or more crosslinking/polymerization promoting agents is an aldehyde, an isocyanate, or a carbodiimide. In another embodiment, the one or more crosslinking/polymerization promoting agents is glutaraldehyde, hexamethylene diisocyanate, or 1-ethyl-3-(3-dimethylamino-propyl) carbodiimide. In another embodiment, the initiator is electromagnetic radiation including ultraviolet light, infrared light, and visible light, or the initiator is ultrasound, magnetism, or heat. In another embodiment, the implant strengthens or reshapes a body tissue that contains one or more extracellular matrix (ECM) proteins.

Most animal cells release materials into the extracellular space, creating a complex meshwork of components such as proteins and carbohydrates called the ECM. A major component of the ECM is the protein collagen. Collagen proteins are modified with carbohydrates, and once they're released from the cell, they assemble into long fibers called collagen fibrils. In the ECM, collagen fibers are interwoven with a class of carbohydrate-bearing proteoglycans, which may be attached to a long polysaccharide backbone. Collagen plays a key role in giving tissues strength and structural integrity. The ECM also contains many other types of proteins and carbohydrates. For example, ECM components include but are not limited to heparan sulfate, chondroitin sulfate, keratan sulfate, hyaluronic acid, elastin, fibronectin and laminin. Any one or more of the ECM components can be crosslinked/polymerized in the presence of one or more crosslinking/polymerization promoting agents to address the loss of strength in various tissues.

In one embodiment, crosslinking/polymerization of ECM components such as collagen, involves the formation of covalent bonds between collagen molecules using crosslinking/polymerization promoting agents, which bind either to a free amine or a carboxyl group of the collagen. The most commonly used chemical crosslinking/polymerization agents are aldehydes (e.g., glutaraldehyde), isocyanates (e.g., hexamethylene diisocyanate [HMDI]), carbodiimides (e.g., 1-ethyl-3-(3-dimethylaminopropyl) carbodiimide [EDC]), and polyethylene glycol (PEG) polymers that can vary in molecular weight, degree of branching, and terminal groups.

In other embodiments, crosslinking/polymerization of ECM components involves the formation of covalent bonds directly between the ECM components using crosslinking/polymerization agents that act as photoreactive agents (e.g., rose bengal, riboflavin), carbohydrates (e.g., ribose, glucose), plant extracts (e.g., genipin, oleuropein, and Myrica rubra). For example, riboflavin and rose bengal serve as a photosensitizers to generate radicals but do not directly get incorporated into the resulting crosslinked polymer. When riboflavin or rose bengal are activated by an initiator, e.g. an energy source such as UVA radiation, oxygen radicals are released followed by formation of new crosslinked covalent bonds directly between collagen, thereby stiffening and shaping the corneal implant.

The monomers and/or oligomers of ECM components can be mixed with one or more chemical agents that promote crosslinking/polymerization. The mixture of monomers and/or oligomers can be incompletely or partially cross-linked/polymerized when the mixture is delivered to an anatomic site, e.g. the cornea of an eye, and can become further crosslinked/polymerized on exposure to an initiator and/or over time. In an embodiment, the mixture can in the form of a fluid, gel or paste, which can be delivered by injection or through a small incision. In another embodiment, the mixture can become crosslinked/polymerized without any additional intervention.

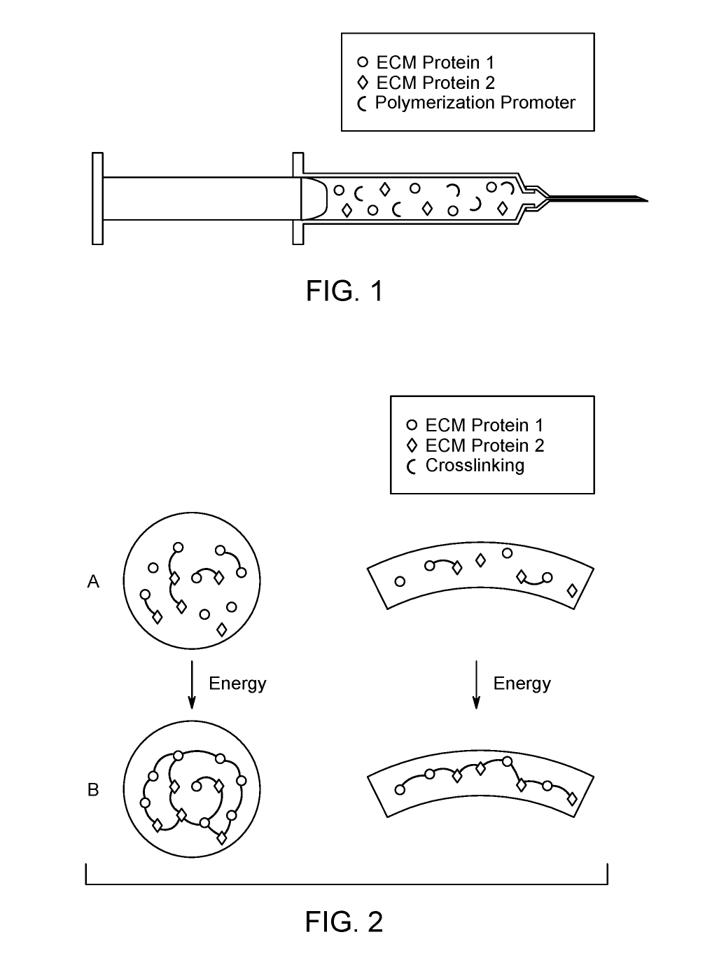

FIG. 1 illustrates an embodiment of a delivery device containing a mixture of ECM components and one or more crosslinking/polymerization promoting agents in a liquid or gel form. This allows it to be injected or otherwise delivered through a small opening in the target tissue. Circles and diamonds represent ECM components and curved lines indicate the crosslinking/polymerization promoting agent.

In other embodiments, the mixture can crosslink/polymerize after exposure to some form of electromagnetic radiation such as ultraviolet light or heat or other energy source such as ultrasound or magnetism.

The polymerization/cross-linking of the compound within the anatomic site can produce a strengthening effect within the tissues and/or change in shape to a therapeutically desirable state. For example, a compound of collagen monomers or oligomers and riboflavin can be delivered to a keratoconus cornea for the purpose of strengthening the cornea. This can be accomplished by first creating a pocket within the cornea which will hold the compound. Ultraviolet radiation is then applied to the cornea to achieve polymerization of the mixture.

In one embodiment, the disclosure provides a corneal implant that is partially crosslinked/polymerized. FIG. 2A illustrates an embodiment of a top view of a corneal implant that is partially crosslinked/polymerized and fully crosslinked/polymerized on exposure to an initiator; and FIG. 2B illustrates an embodiment of a cross-sectional view of a corneal implant that is partially crosslinked/polymerized and fully crosslinked/polymerized on exposure to an initiator. By exposing the implant to more energy, the implant becomes more crosslinked/polymerized. In the case of a corneal implant, ambient light e.g. sunlight can be the source of energy to promote crosslinking/polymerization because the cornea is exposed to ambient light. Circles and diamonds represent ECM proteins. The curved lines between the ECM proteins indicate that the ECM proteins have been crosslinked or polymerized. Note how the exposure of the implant to energy results in more crosslinking/polymerization.

In another embodiment, eye bags can be treated by exposure to an implant containing a mixture of one or more ECM components and one or more crosslinking/polymerization agents, wherein the orbital septum weakens with age and allows anterior herniation of the orbital fat. This results in a characteristically older appearance, which is often considered undesirable. In accordance with the teachings of this invention, the mixture can be delivered to the region of the orbital septum by injection and an energy source such as ultrasound and the like, could be used to promote crosslinking/polymerization.

In another embodiment, an implant including one or more ECM components (e.g., monomers and/or oligomers) and one or more polymerization/crosslinking agents can be implanted within an anatomic site to produce a strengthening effect within the tissues and/or change in shape to a therapeutically desirable state. The implant should be flexible and reversibly deformable. For example, an implant composed of collagen and a crosslinking agent such as riboflavin could be implanted within a cornea that has keratoconus within a corneal pocket. Ultraviolet light can be used to crosslink the collagen within the implant to make the implant stiffer, which will strengthen the recipient cornea and change the shape of the cornea to a more optically useful state.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of sonic embodiments of the invention shall not be construed as an admission that such reference is available as prior art to the present invention.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

What is claimed is:

1. An implant, comprising:
one or more extracellular matrix (ECM) components; and
one or more crosslinking/polymerization promoting agents, wherein the one or more ECM components undergo crosslinking/polymerization on exposure to an initiator.

2. The implant of claim 1, wherein the implant is a corneal implant.

3. The implant of claim 1, wherein the ECM components and the one or more crosslinking/polymerization promoting agents is in a liquid or gel form.

4. The implant of claim 1, wherein the one or more extracellular matrix (ECM) components undergo crosslinking/polymerization with the one or more crosslinking/polymerization promoting agents on exposure to an initiator.

5. The implant of claim 1, wherein the one or more ECM components is an ECM protein or a ECM carbohydrate, or a combination thereof.

6. The implant of claim 5, wherein the one or more ECM components is collagen, heparan sulfate, chondroitin sulfate, keratan sulfate, hyaluronic acid, elastin, fibronectin, laminin, or a combination thereof.

7. The implant of claim 1, wherein the one or more crosslinking/polymerization promoting agents is a photoreactive agent, a carbohydrate, or a plant extract.

8. The implant of claim 7, wherein the photoreactive agent is riboflavin or rose bengal and the initiator is ultraviolet light.

9. The implant of claim 4, wherein the one or more crosslinking/polymerization promoting agents is an aldehyde, an isocyanate, or a carbodiimide.

10. The implant of claim 9, wherein the one or more crosslinking/polymerization promoting agents is glutaraldehyde, hexamethylene diisocyanate, or 1-ethyl-3-(3-dimethylamino-propyl) carbodiimide.

11. The implant of claim 1, wherein the initiator is electromagnetic radiation including ultraviolet light, infrared light, and visible light, or the initiator is ultrasound, magnetism, or heat.

12. The implant of claim 1, wherein the implant strengthens or reshapes a body tissue that contains one or more extracellular matrix (ECM) proteins.

13. A method of preparing a corneal implant, comprising:
mixing one or more extracellular matrix (ECM) components with one or more crosslinking/polymerization promoting agents; and
inserting the mixture into an eye of a patient in need thereof, wherein the ECM proteins undergo crosslinking with the one or more crosslinking/polymerization promoting chemical agents on exposure to an initiator.

14. The method of preparing a corneal implant of claim 13, wherein the ECM components and the one or more crosslinking/polymerization promoting agents is in a liquid or gel form.

15. The method of preparing a corneal implant of claim 13, wherein the one or more extracellular matrix (ECM) components undergo crosslinking/polymerization with the one or more crosslinking/polymerization promoting agents on exposure to an initiator.

16. The method of preparing a corneal implant of claim 13, wherein the one or more ECM components is an ECM protein or a ECM carbohydrate, or a combination thereof.

17. The method of preparing a corneal implant of claim 16, wherein the one or more ECM components is collagen, heparan sulfate, chondroitin sulfate, keratan sulfate, hyaluronic acid, elastin, fibronectin, laminin, or a combination thereof.

18. The method of preparing a corneal implant of claim 13, wherein the one or more crosslinking/polymerization promoting agents is a photoreactive agent, a carbohydrate, or a plant extract.

19. The method of preparing a corneal implant of claim 18, wherein the photoreactive agent is riboflavin or rose bengal and the initiator is ultraviolet light.

20. The method of preparing a corneal implant of claim 13, wherein the one or more crosslinking/polymerization promoting agents is an aldehyde, an isocyanate, or a carbodiimide.

21. The method of preparing a corneal implant of claim 20, wherein the one or more crosslinking/polymerization promoting agents is glutaraldehyde, hexamethylene diisocyanate, or 1-ethyl-3-(3-dimethylamino-propyl) carbodiimide.

22. The method of preparing a conical implant of claim 13, wherein the initiator is electromagnetic radiation including ultraviolet light, infrared light, and visible light, or the initiator is ultrasound, magnetism, or heat.

23. The method of preparing a corneal implant of claim 13, wherein the implant strengthens or reshapes a body tissue that contains one or more extracellular matrix (ECM) proteins.
